(19) United States
(12) Patent Application Publication
(10) Pub. No.: US 2010/0234411 A1
(43) Pub. Date: Sep. 16, 2010

(54) New Combination for the Treatment of Respiratory Diseases

(75) Inventors: Yunhai Cui, Biberach (DE); Wolfgang Wienen, Biberach (DE); Michael P. Pieper, Biberach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERMATIONAL GMBH, Ingelheim am Rhein (DE)

(21) Appl. No.: 12/669,233

(22) PCT Filed: Jul. 14, 2008
(86) PCT No.: PCT/EP08/59174

§ 371 Date: Apr. 12, 2010

(30) Foreign Application Priority Data
Jul. 18, 2007 (EP) 07112711.2

(51) Int. Cl. A61K 31/46; A61K 31/4184; A61K 31/439; A61K 31/40; A61K 31/138; A61K 31/14; A61P 11/00

(52) U.S. Cl. 514/278; 514/394; 514/294; 514/305; 514/304; 514/424; 514/657; 514/643

(57) ABSTRACT

The present invention relates to novel pharmaceutical compositions based on telmisartan 1 and anticholinergics 2 processes for preparing them and their use for the treatment of respiratory diseases.

The present invention relates to novel pharmaceutical compositions based on telmisartan 1 and anticholinergics 2, processes for preparing them and their use for the treatment of respiratory diseases.

### DESCRIPTION OF THE INVENTION

The present invention relates to novel pharmaceutical compositions based on telmisartan 1, optionally in form of the salts, solvates or hydrates thereof, and anticholinergics 2, optionally in the form of the solvates or hydrates thereof, processes for preparing them and their use for the treatment of respiratory diseases.

Telmisartan 1 can be present in the compositions according to the invention in the form of suitable pharmacologically acceptable salts. These pharmacologically acceptable salts include alkali metal salts, e.g., sodium or potassium salts; alkaline earth metal salts, e.g., calcium or magnesium salts; and salts formed with suitable organic ligands, e.g., quaternary ammonium salts.

Telmisartan 1 could also be present in the compositions according to the invention in the form of pharmacologically acceptable acid addition salts. These acid addition salts may be selected from the group comprising the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate. Of the above-mentioned acid addition salts, the salts of hydrochloric acid, methanesulphonic acid, benzoic acid and acetic acid are particularly preferred according to the invention.

In the medicament combinations according to the invention the anticholinergic 2 is preferably selected from among the tiotropium salts 2.1), oxitropium salts (22), flutropium salts 2.3), ipratropium salts (M), glycopyrronium salts 2.5), trospium salts (2.6) and the compounds of formulae 2.7 to 2.13.

In the above-mentioned salts 2.1 to 2.6 the cations tiotropium, oxitropium, flutropium, ipratropium, glycopyrronium and trospium are the pharmacologically active constituents. Explicit references to the above-mentioned cations are indicated by the numerals 2.1′ to 2.6′. Each reference to the above-mentioned salts 2.1 to 2.6 naturally includes a reference to the corresponding cations tiotropium (2.1′), oxitropium (2.2′), flutropium (2.3′), ipratropium (2.4′), glycopyrronium (2.5′) and trospium (2.6′).

By the salts 2.1 to 2.6 are meant according to the invention those compounds which contain in addition to the cations tiotropium (2.1′), oxitropium (2.2′), flutropium (2.3′), ipratropium (2.4′), glycopyrronium (2.5′) and trospium (2.6′) as counter-ion (anion) chloride, bromide, iodide, sulphate, phosphate, methanesulphonate, nitrate, maleate, acetate, citrate, fumarate, tartrate, oxalate, succinate, benzoate or p-toluenesulphonate contain, while the chloride, bromide, iodide, sulphate, methanesulphonate or p-toluenesulphonate are preferred as counter-ions. Of all the salts the chloride, bromide, iodide and methanesulphonate are particularly preferred.

In the case of the trospium salts (2.6) the chloride is particularly preferred. Of the other salts 2.1 to 2.5 the methanesulphonates and bromides are of particular importance.

Of particular importance are medicament combinations which contain tiotropium salts (2.1), oxitropium salts 2.2) or ipratropium salts (2.4), while the respective bromides are particularly important according to the invention. Of particular importance is the tiotropium bromide (2.1). The above-mentioned salts may optionally be present in the medicament combinations according to the invention in the form of their solvates or hydrates, preferably in the form of their hydrates. In the case of tiotropium bromide the medicament combinations according to the invention preferably contain this in the form of the crystalline tiotropium bromide monohydrate, which is known from WO 02/30928. If the tiotropium bromide is used in anhydrous form in the medicament combinations according to the invention, it is preferable to use the anhydrous crystalline tiotropium bromide which is known from WO 03/000265.

The above-mentioned anticholinergics optionally have chiral carbon centres. In this case the medicament combinations according to the invention may contain the anticholinergics in the form of their enantiomers, mixtures of enantiomers or racemates, while enantiomerically pure anticholinergics as for instance R,R-glycopyrrolate (2.5) are preferably used.

In another preferred embodiment of the present invention the anticholinergics 2 contained in the medicament combinations according to the invention are selected from the salts of formula 2.7

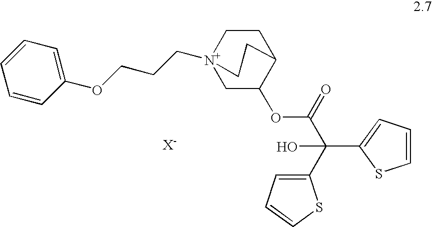

wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
   optionally in the form of the racemates, enantiomers or hydrates thereof.

Preferred medicament combinations contain salts of formula 2.7, wherein
X⁻ denotes an anion with a single negative charge, preferably an anion selected from among the fluoride, chloride, bromide, methanesulphonate and p-toluenesulphonate, preferably bromide,
   optionally in the form of the racemates, enantiomers or hydrates thereof.

Preferred medicament combinations contain salts of formula 2.7, wherein
X⁻ denotes an anion with a single negative charge, preferably an anion selected from among the chloride, bromide and methanesulphonate, preferably bromide,
   optionally in the form of the racemates, enantiomers or hydrates thereof.

Particularly preferred medicament combinations contain the compound of formula 2.7 in the form of the bromide.

Of particular importance are those medicament combinations which contain the enantiomers of formula 2.7-en

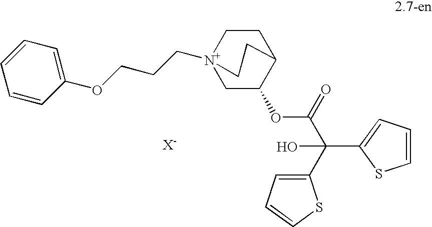

wherein X⁻ may have the above-mentioned meanings.

In another preferred embodiment of the present invention the anticholinergics 2 contained in the medicament combinations according to the invention are selected from the salts of formula 2.8

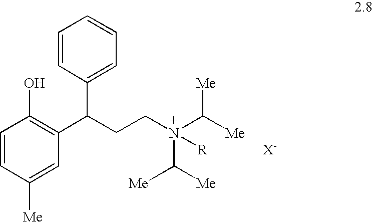

wherein R denotes either methyl (2.8.1) or ethyl (2.8.2) and wherein X⁻ may have the above-mentioned meanings. In an alternative embodiment the compound of formula 2.8 is present in the form of the free base 2.8-base

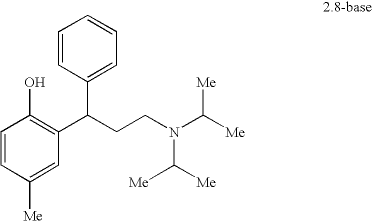

The medicament combinations according to the invention may contain the anticholinergic of formula 2.8 (or 2.8-base) in the form of the enantiomers, mixtures of enantiomers or racemates thereof. Preferably the anticholinergics of formula 2.8 (or 2.8-base) are present in the form of their R-enantiomers.

In another preferred embodiment of the present invention the anticholinergics 2 contained in the medicament combinations according to the invention are selected from the compounds of formula 2.9

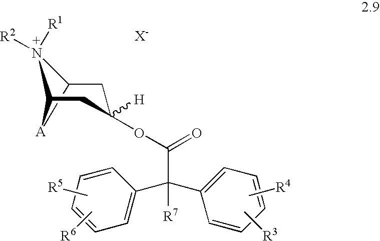

wherein
A denotes a double-bonded group selected from the groups

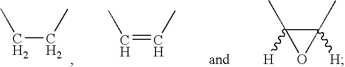

X⁻ denotes one of the above-mentioned anions with a single negative charge, preferably chloride, bromide or methanesulphonate,
R¹ and R² which may be identical or different denote a group selected from methyl, ethyl, n-propyl and iso-propyl, which may optionally be substituted by hydroxy or fluorine, preferably unsubstituted methyl;
R³, R⁴, R⁵ and R⁶, which may be identical or different denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, —O—CH₂—F, —O—CH₂—CH₂—F, —CH₂—OH, —CH₂—CH₂—OH, CF₃, —CH₂—OMe, —CH₂—CH₂—OMe, —CH₂—OEt, —CH₂—CH₂—OEt, —O—COMe, —O—COEt, —O—COCF₃, —O—COCF₃, fluorine, chlorine or bromine.

The compounds of formula 2.9 are known in the art (WO 02/32899).

Within the scope of the medicament combinations according to the invention preferred compounds of formula 2.9 are those wherein
X⁻ denotes bromide;
R¹ and R² which may be identical or different, denote methyl or ethyl, preferably methyl;
R³, R⁴, R⁵ and R⁶, which may be identical or different, denote hydrogen, methyl, methyloxy, chlorine or fluorine;
R⁷ denotes hydrogen, methyl or fluorine.

Of particular importance are medicament combinations which contain compounds of formula 2.9, wherein
A denotes a double-bonded group selected from

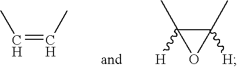

Of particular importance are those medicament combinations which contain in addition to a compound 1 one of the following compounds of formula 2.9:
tropenol 2,2-diphenylpropionate methobromide (2.9.1),
scopine 2,2-diphenylpropionate methobromide (2.9.2),
scopine 2-fluoro-2,2-diphenylacetate methobromide (2.9.3),
tropenol 2-fluoro-2,2-diphenylacetate methobromide (2.9.4);

The compounds of formula 2.9 may optionally in the form of the enantiomers, mixtures of enantiomers or racemates thereof, as well as optionally in the form of the hydrates and/or solvates thereof.

In another preferred embodiment of the present invention the anticholinergics 2 contained in the medicament combinations according to the invention are selected from the compounds of formula 2.10

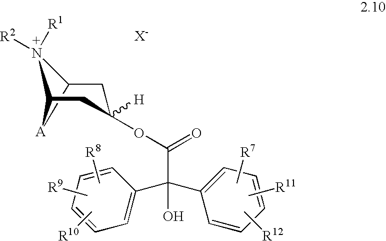

wherein

A, X⁻, R¹ and R² may have the meanings given above and wherein

R⁷, R⁸, R⁹, R¹⁰, R¹¹ and R¹², which may be identical or different, denote hydrogen, methyl, ethyl, methyloxy, ethyloxy, hydroxy, fluorine, chlorine, bromine, CN, CF₃ or NO₂, while at least one of the groups R⁷, R⁸, R⁹, R¹⁰, R¹¹ and R¹² may not be hydrogen.

The compounds of formula 2.10 are known in the art (WO 02/32898).

Within the scope of the medicament combinations according to the invention preferred compounds of formula 2.10 are those wherein
A denotes a double-bonded group selected from

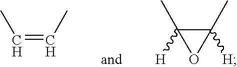

X⁻ bromide;
R¹ and R² which may be identical or different, denote methyl or ethyl, preferably methyl;
R⁷, R⁸, R⁹, R¹⁰, R¹¹ and R¹², which may be identical or different, denote hydrogen, fluorine, chlorine or bromine, preferably fluorine, while at least one of the groups R⁷, R⁸, R⁹, R¹⁰, R¹¹ and R¹² may not be hydrogen.

Of particular importance are those medicament combinations which contain in addition to a compound 1 one of the following compounds of formula 2.10:
tropenol 3,3′,4,4′-tetrafluorobenzilate methobromide (2.10.1),
scopine 3,3′,4,4′-tetrafluorobenzilate methobromide (2.10.2),
tropenol 4,4′-difluorobenzilate methobromide (2.10.3),
scopine 4,4′-difluorobenzilate methobromide (2.10.4),
tropenol 3,3′-difluorobenzilate methobromide (2.10.5),
scopine 3,3′-difluorobenzilate methobromide (2.10.6).

The compounds of formula 2.10 may optionally be presenting the form of the enantiomers, mixtures of enantiomers or racemates thereof, as well as optionally in the form of the hydrates and/or solvates thereof.

In another preferred embodiment of the present invention the anticholinergics 2 contained in the medicament combinations according to the invention are selected from the compounds of formula 2.11

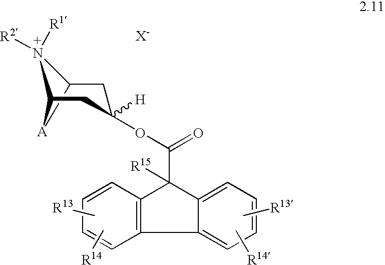

wherein
A and X⁻ may have the meanings given above and wherein
R¹⁵ denotes hydrogen, hydroxy, methyl, ethyl, —CF₃, CHF₂ or fluorine;
R^{1′} and R^{2′} which may be identical or different, denote C₁-C₅-alkyl, which may optionally be substituted by C₃-C₆-cycloalkyl, hydroxy or halogen,
   or
   R^{1′} and R^{2′} together denote a —C₃-C₅-alkylene bridge;
R¹³, R¹⁴, R^{13′} and R^{14′} which may be identical or different, denote hydrogen, —C₁-C₄-alkyl,
   —C₁-C₄-alkyloxy, hydroxy, —CF₃, —CHF₂, CN, NO₂ or halogen.

The compounds of formula 2.11 are known in the art (WO 03/064419).

Within the scope of the medicament combinations according to the invention preferred compounds of formula 2.11 are those wherein
A denotes a double-bonded group selected from

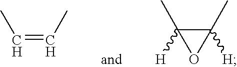

X⁻ denotes an anion selected from chloride, bromide and methanesulphonate, preferably bromide;
R¹⁵ denotes hydroxy, methyl or fluorine, preferably methyl or hydroxy;

R^{1′} and R^{2′} which may be identical or different, denote methyl or ethyl, preferably methyl;
R¹³, R¹⁴, R^{13′} and R^{14′} which may be identical or different, denote hydrogen, —CF₃, —CHF₂ or fluorine, preferably hydrogen or fluorine.

Within the scope of the medicament combinations according to the invention particularly preferred compounds of formula 2.11 are those wherein
A denotes a double-bonded group selected from

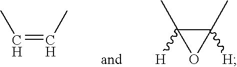

X⁻ denotes bromide;
R¹⁵ denotes hydroxy or methyl, preferably methyl;
R^{1′} and R^{2′} which may be identical or different, denote methyl or ethyl, preferably methyl;
R¹³, R¹⁴, R^{13′} and R^{14′} which may be identical or different, denote hydrogen or fluorine.

Of particular importance are those medicament combinations which contain in addition to a compound 1 one of the following compounds of formula 2.11:
tropenol 9-hydroxy-fluorene-9-carboxylate methobromide (2.12a.1);
tropenol 9-fluoro-fluorene-9-carboxylate methobromide (2.12a.2);
scopine 9-hydroxy-fluorene-9-carboxylate methobromide (2.12a.3);
scopine 9-fluoro-fluorene-9-carboxylate methobromide (2.12a.4);
tropenol 9-methyl-fluorene-9-carboxylate methobromide (2.12a.5);
scopine 9-methyl-fluorene-9-carboxylate methobromide (2.12a.6);

The compounds of formula 2.11 may optionally be present in the form of the enantiomers, mixtures of enantiomers or racemates thereof, as well as optionally in the form of the hydrates and/or solvates thereof.

In another preferred embodiment of the present invention the anticholinergics 2 contained in the medicament combinations according to the invention are selected from the compounds of formula 2.12

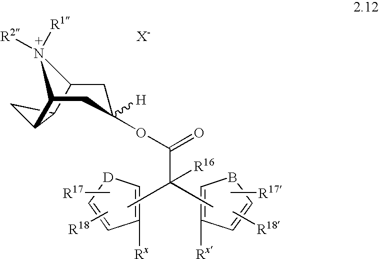

wherein X⁻ may have the meanings given above and wherein
D and B which may be identical or different, are preferably identical and denote O, S, NH, CH₂, CH═CH or
   N(C₁-C₄-alkyl);
R¹⁶ denotes hydrogen, hydroxy, C₁-C₄ alkyl —C₁-C₄-alkyloxy, —C₁-C₄-alkylene-halogen, —O—C₁-C₄-alkylene-halogen, —C₁-C₄-alkylene-OH, —CF₃, CHF₂, —C₁-C₄-alkylene-C₁-C₄-alkyloxy, —C₄-alkylene-halogen, —C₁-C₄-alkylene-C₃-C₆-cycloalkyl, —O—COCF₃ or halogen;
R^{1″} and R^{2″} which may be identical or different, denote —C₁-C₅-alkyl, which may optionally be substituted by —C₃-C₆-cycloalkyl, hydroxy or halogen,
   or
   R^{1″} and R^{2″} together denote a —C₃-C₅-alkylene bridge;
R¹⁷, R¹⁸, R^{17′} and R^{18′}, which may be identical or different, denote hydrogen, —C₁-C₄-alkyl, C₁-C₄ alkyloxy hydroxy, —CF₃, —CHF₂, CN, NO₂ or halogen;
R^{x} and R^{x′} which may be identical or different, denote hydrogen, —C₁-C₄-alkyl, —C₁-C₄-alkyloxy, hydroxy, —CF₃, —CHF₂, CN, NO₂ or halogen,
   or
R^{x} and R^{x′} together denote a single bond or one of the double-bonded groups O, S, NH, CH₂, CH₂—CH₂, N(C₁-C₄-alkyl), CH(C₁-C₄-alkyl) and —C(C₁-C₄-alkyl)₂.

The compounds of formula 2.12 are known in the art (WO 03/064418).

Within the scope of the medicament combinations according to the invention preferred compounds of formula 2.12 are those wherein
X⁻ denotes chloride, bromide or methanesulphonate, preferably bromide;
D and B which may be identical or different, are preferably identical and denote O, S, NH or CH═CH;
R¹⁶ denotes hydrogen, hydroxy, —C₁-C₄-alkyl, —C₁-C₄-alkyloxy, —CF₃, —CHF₂, fluorine, chlorine or bromine;
R^{1″} and R^{2″} which may be identical or different, denote C₁-C₄-alkyl, which may optionally be substituted by hydroxy, fluorine, chlorine or bromine,
   or
R^{1″} and R^{2″} together denote a —C₃-C₄-alkylene bridge;
R¹⁷, R¹⁸, R^{17′} and R^{18′}, which may be identical or different, denote hydrogen, C₁-C₄-alkyl, C₁-C₄-alkyloxy, hydroxy, —CF₃, —CHF₂, CN, NO₂, fluorine, chlorine or bromine;
R^{x} and R^{x′} which may be identical or different, denote hydrogen, C₁-C₄-alkyl, C₁-C₄-alkyloxy, hydroxy, —CF₃, —CHF₂, CN, NO₂, fluorine, chlorine or bromine, or
R^{x} and R^{x′} together denote a single bond or a double-bonded group selected from O, S, NH— and CH₂.

Within the scope of the medicament combinations according to the invention particularly preferred compounds of formula 2.12 are those wherein
X⁻ denotes chloride, bromide, or methanesulphonate, preferably bromide;
D and B which may be identical or different, preferably identical, denote S or CH═CH;
R¹⁶ denotes hydrogen, hydroxy or methyl;
R^{1″} and R^{2″} which may be identical or different, denote methyl or ethyl;
R¹⁷, R¹⁸, R^{17′} and R^{18′}, which may be identical or different, denote hydrogen, —CF₃ or fluorine, preferably hydrogen;
R^{x} and R^{x′} which may be identical or different, denote hydrogen, —CF₃ or fluorine, preferably hydrogen, or
R^{x} and R^{x′} together denote a single bond or —O.

Within the scope of the medicament combinations according to the invention, other particularly preferred compounds of formula 2.12 are those wherein
X⁻ denotes bromide;
D and B denotes —CH═CH—;
R¹⁶ denotes hydrogen, hydroxy or methyl;
R^{1″} and R^{2″} denotes methyl;
R¹⁷, R¹⁸, R^{17′} and R^{18′}, which may be identical or different, denote hydrogen or fluorine, preferably hydrogen;
R^{x} and R^{x′} which may be identical or different, denote hydrogen or fluorine, preferably hydrogen, or
R^{x} and R^{x′} together denote a single bond or the group —O.

Of particular importance are those medicament combinations which contain in addition to a compound 1 one of the following compounds of formula 2.12:
cyclopropyltropine benzilate methobromide (2.12.1);
cyclopropyltropine 2,2-diphenylpropionate methobromide (2.12.2);
cyclopropyltropine 9-hydroxy-xanthene-9-carboxylate methobromide (2.12.3);
cyclopropyltropine 9-methyl-fluorene-9-carboxylate methobromide (2.12.4);
cyclopropyltropine 9-methyl-xanthene-9-carboxylate methobromide (2.12.5);
cyclopropyltropine 9-hydroxy-fluorene-9-carboxylate methobromide (2.12.6);
cyclopropyltropine methyl 4,4′-difluorobenzilate methobromide (2.12.7).

The compounds of formula 2.12 may optionally be present in the form of the enantiomers, mixtures of enantiomers or racemates thereof, as well as optionally in the form of the hydrates and/or solvates thereof.

In another preferred embodiment of the present invention the anticholinergics 2 contained in the medicament combinations according to the invention are selected from the compounds of formula 2.13

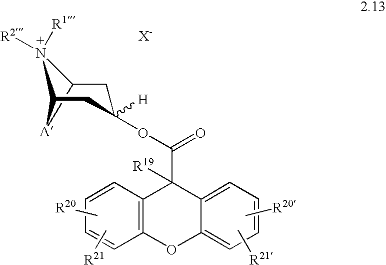

wherein X⁻ may have the meanings given above and wherein
A′ denotes a double-bonded group selected from

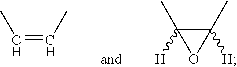

R¹⁹ denotes hydroxy, methyl, hydroxymethyl, ethyl, —CF₃, CHF₂ or fluorine;
R^{1″′} and R^{2′″} which may be identical or different, denote C₁-C₅-alkyl, which may optionally be substituted by C₃-C₆-cycloalkyl, hydroxy or halogen,
   or
R^{1′″} and R^{2′″} together denote a —C₃-C₅-alkylene bridge;
R²⁰, R²¹, R^{20′} and R^{21′} which may be identical or different, denote hydrogen, —C₁-C₄-alkyl, —C₁-C₄-alkyloxy, hydroxy, —CF₃, —CHF₂, CN, NO₂ or halogen.

The compounds of formula 2.13 are known in the art (WO 03/064417).

Within the scope of the medicament combinations according to the invention preferred compounds of formula 2.13 are those wherein
A′ denotes a double-bonded group selected from

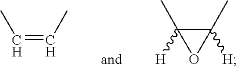

X⁻ denotes chloride, bromide or methanesulphnat, preferably bromide;
R¹⁹ denotes hydroxy or methyl;
R^{1′″} and R^{2′″} which may be identical or different, denote methyl or ethyl, preferably methyl;
R²⁰, R²¹, R^{20′} and R^{21′} which may be identical or different, denote hydrogen, —CF₃, —CHF₂ or fluorine, preferably hydrogen or fluorine.

Within the scope of the medicament combinations according to the invention particularly preferred compounds of formula 2.13 are those wherein
A′ denotes a double-bonded group selected from

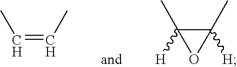

X⁻ denotes bromide;
R¹⁹ denotes hydroxy or methyl, preferably methyl;
R^{1′″} and R^{2′″} which may be identical or different, denote methyl or ethyl, preferably methyl;
R³, R⁴, R^{3′} and R^{4′} which may be identical or different, denote hydrogen or fluorine.

Of particular importance are those medicament combinations which contain in addition to a compound 1 one of the following compounds of formula 2.13:
tropenol 9-hydroxy-xanthene-9-carboxylate methobromide (2.12c.1);
scopine 9-hydroxy-xanthene-9-carboxylate methobromide (2.12c.2);
tropenol 9-methyl-xanthene-9-carboxylate methobromide (2.12c.3);
scopine 9-methyl-xanthene-9-carboxylate methobromide (2.12c.4);
tropenol 9-ethyl-xanthene-9-carboxylate methobromide (2.12c.5);
tropenol 9-difluoromethyl-xanthene-9-carboxylate methobromide (2.12c.6);
scopine 9-hydroxymethyl-xanthene-9-carboxylate methobromide (2.12c.7).

The compounds of formula 2.13 may optionally be present in the form of the enantiomers, mixtures of enantiomers or racemates thereof, as well as optionally in the form of the hydrates and/or solvates thereof.

Within the scope of the present invention any reference to anticholinergics 2′ is to be taken as a reference to the pharmacologically active cations of the various salts. These cations are tiotropium (2.1′), oxitropium (2.2′), flutropium (2.3′), ipratropium (2.4′), glycopyrronium (2.5′) trospium (2.6′) and the cations shown below:

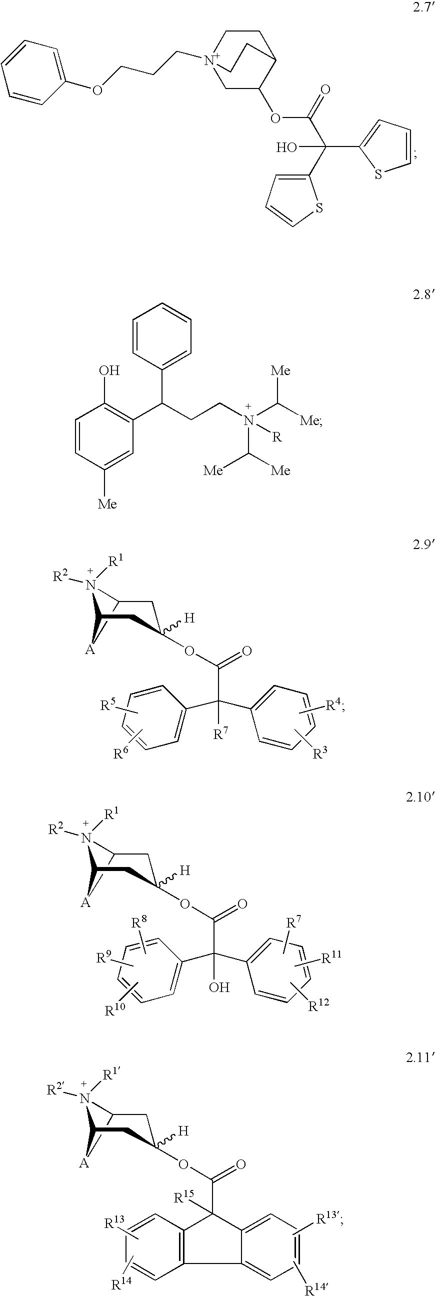

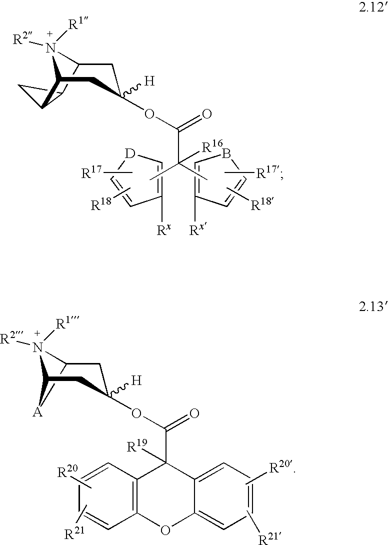

The pharmaceutical compositions according to the invention may contain besides telmisartan 1 and the anticholinergic 2 another active ingredient. This additional active ingredient may be selected from the group of PDEIV inhibitor, steroids, LTD4 antagonist, or for instance betamimetics.

In a yet another preferred embodiment the medicament combinations according to the invention contain as the anticholinergic 2 one or more, preferably one compound selected from the group consisting of 2.1, 2.4, 2.5, 2.7, 2.9.1, 2.9.2, 2.12.1 and 2.12.2, more preferably selected from among 2.1, 2.5, 2.7, 2.9.1 and 2.9.2.

Unless otherwise stated, the alkyl groups are straight-chained or branched alkyl groups having 1 to 4 carbon atoms. The following are mentioned by way of example: methyl, ethyl, propyl or butyl. In some cases the abbreviations Me, Et, Prop or Bu are used to denote the groups methyl, ethyl, propyl or butyl. Unless otherwise stated, the definitions propyl and butyl include all the possible isomeric forms of the groups in question. Thus, for example, propyl includes n-propyl and iso-propyl, butyl includes iso-butyl, sec.butyl and tert.-butyl, etc.

Unless otherwise stated, the cycloalkyl groups are alicyclic groups with 3 to 6 carbon atoms. They are the cyclopropyl, cyclobutyl, cyclopentyl and cyclohexyl groups. Cyclopropyl is particularly important within the scope of the present invention.

Unless otherwise stated, the alkylene groups are branched and unbranched double-bonded alkyl bridges with 1 to 4 carbon atoms. Examples include: methylene, ethylene, propylene or butylene.

Unless otherwise stated, the alkylene-halogen groups are branched and unbranched double-bonded alkyl bridges with 1 to 4 carbon atoms which are mono-, di- or trisubstituted, preferably disubstituted, by a halogen. Accordingly, unless otherwise stated, alkylene-OH-groups are branched and unbranched double-bonded alkyl bridges with 1 to 4 carbon atoms which are mono-, di- or trisubstituted, preferably monosubstituted, by a hydroxy.

Unless otherwise stated, the term alkyloxy groups denotes branched and unbranched alkyl groups with 1 to 4 carbon atoms which are linked via an oxygen atom. Examples include: methyloxy, ethyloxy, propyloxy or butyloxy. In some cases the abbreviations MeO, EtO, PropO or BuO may be used to denote the methyloxy, ethyloxy, propyloxy or butyloxy groups. Unless otherwise stated, the definitions propyloxy and butyloxy include all the possible isomeric forms of the groups in question. Thus, for example, propyloxy includes n-propyloxy and iso-propyloxy, butyloxy includes iso-butyloxy, sec.butyloxy and tert.-butyloxy, etc. In some cases the term alkoxy may be used instead of alkyloxy within the scope of the present invention. The groups methyloxy, ethyloxy, propyloxy or butyloxy may therefore also be referred to by the names methoxy, ethoxy, propoxy or butoxy.

Unless otherwise stated, the term alkylene-alkyloxy refers to branched and unbranched double-bonded alkyl bridges with 1 to 4 carbon atoms which are mono-, di- or trisubstituted, preferably monosubstituted, by an alkyloxy group.

Unless otherwise stated, the term —O—CO-alkyl groups refers to branched and unbranched alkyl groups with 1 to 4 carbon atoms which are linked by an ester group. The alkyl groups are attached directly to the carbonyl carbon of the ester group. The term —O—CO-alkyl-halogen should be understood analogously. The group —O—CO—CF₃ denotes trifluoroacetate.

Halogen within the scope of the present invention denotes fluorine, chlorine, bromine or iodine. Unless stated otherwise, fluorine and bromine are the preferred halogens. The group CO denotes a carbonyl group.

Within the scope of the present invention by a pharmaceutical combination of components 1 and 2 is meant the joint administration of the active substances in a single preparation or formulation or the separate administration of the active substances in separate formulations. If the active substances are administered in separate formulations, this separate administration may be done simultaneously or at different times, i.e. successively. Successive administration is to be understood as administration of 1 and 2 in different formulations. As an example 1 may be administered orally, and 2 by way of inhalation. Preferably 1 is administered once or twice daily, preferably once daily.

Preferably 2 is also administered once or twice daily, preferably once daily. 1 is preferably administered once daily either in the morning or in the evening. 2 is also preferably administered once daily either in the morning or in the evening. Preferred administration of 2 is in the morning. Successive administration of 1 and 2 may occur that way, that 1 is administered for instance in the morning, and 2 is administered either shortly before the administration of 1 or shortly thereafter. Successive administration within the meaning of the invention in hand does also include a dose regimen in which administration of 2 occurs once daily in the morning and administration of 1 occurs once daily in the evening.

In one aspect the present invention relates to the above-mentioned medicament combinations which contain in addition to therapeutically effective amounts of 1, optionally also 2 and a pharmaceutically acceptable carrier. In one aspect the present invention relates to the above-mentioned pharmaceutical compositions which do not contain a pharmaceutically acceptable carrier in addition to therapeutically effective amounts of 1 and 2.

The present invention also relates to the use of therapeutically effective amounts of the active substances 1 for preparing a pharmaceutical composition also containing one or more, preferably one active substance 2 for the treatment of inflammatory and obstructive respiratory complaints, for inhibiting premature labour in midwifery (tocolysis), for restoring sinus rhythm in the heart in atrioventricular block, for correcting bradycardic heart rhythm disorders (antiarrhythmic), for treating circulatory shock (vasodilatation and increasing the heart volume) as well as for the treatment of skin irritations and inflammation.

In a preferred aspect the present invention relates to the use of therapeutically effective amounts of the telmisartan 1 for preparing a pharmaceutical composition also containing one or more, preferably one, active substance 2 for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the medicament combinations according to the invention are used as specified above for preparing a pharmaceutical composition for the treatment of obstructive pulmonary diseases selected from among bronchial asthma, paediatric asthma, severe asthma, acute asthma attacks, chronic bronchitis and COPD (chronic obstructive pulmonary disease), while it is particularly preferable according to the invention to use them for preparing a pharmaceutical composition for the treatment of bronchial asthma and COPD.

It is also preferable to use the medicament combinations according to the invention for preparing a pharmaceutical composition for the treatment of pulmonary emphysema which has its origins in COPD (chronic obstructive pulmonary disease) or α1-proteinase inhibitor deficiency.

It is also preferable to use the medicament combinations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the medicament combinations according to the invention for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

It is also preferable to use the medicament combinations according to the invention for preparing a pharmaceutical composition for the treatment of cystic fibrosis or mucoviscidosis.

It is also preferable to use the medicament combinations according to the invention for preparing a pharmaceutical composition for the treatment of bronchitis, such as for example bronchitis caused by bacterial or viral infection, allergic bronchitis and toxic bronchitis.

It is also preferable to use the medicament combinations according to the invention for preparing a pharmaceutical composition for the treatment of bronchiectasis.

It is also preferable to use the medicament combinations according to the invention for preparing a pharmaceutical composition for the treatment of pulmonary hypertension.

It is also preferable to use the medicament combinations according to the invention for preparing a pharmaceutical composition for the treatment of ARDS (adult respiratory distress syndrome).

It is also preferable to use the medicament combinations according to the invention for preparing a pharmaceutical composition for the treatment of pulmonary oedema, for example toxic pulmonary oedema after aspiration or inhalation of toxic substances and foreign substances.

It is particularly preferable to use the compounds detailed above for preparing a pharmaceutical composition for the treatment of asthma or COPD. Also of particular importance is the above-mentioned use of medicament combinations according to the invention for preparing a pharmaceutical composition for once-a-day treatment of inflammatory and obstructive respiratory complaints, particularly for the once-a-day treatment of asthma or COPD.

The present invention also relates to the use of therapeutically effective amounts of an telmisartan 1 in combination with therapeutically effective amounts of active substance 2 for preparing a pharmaceutical composition for the treatment of one of the above-mentioned diseases.

The present invention also relates to a process for treating one of the above-mentioned diseases, which is characterised in that therapeutically effective amounts of telmisartan 1 are administered in combination with therapeutically effective amounts of active substance 2.

The present invention also relates to a method for the treatment of one or several diseases mentioned hereinbefore, characterized in that therapeutically effective amounts of telmisartan 1 are administered to a patient in combination with therapeutically effective amounts of active substance 2.

The present invention also relates to a method for the treatment of one or several diseases mentioned hereinbefore, characterized in that therapeutically effective amounts of telmisartan 1 are administered to a patient which is under medical treatment with therapeutically effective amounts of active substance 2.

The present invention also relates to a method for the treatment of one or several diseases mentioned hereinbefore, characterized in that therapeutically effective amounts of an anticholinergic 2 are administered to a patient which is under medical treatment with therapeutically effective amounts of telmisartan 1.

Within the scope of the instant invention for example, 1-200 mg telmisartan 1 are administered per single dose. Preferably, amounts of 1 are administered such that each single dose contains 10-180 mg, preferably 15-140 mg, particularly preferably 20-100 mg of 1. For example and without restricting the present invention thereto, 20 mg, 25 mg, 30 mg, 40 mg, 45 mg, 50 mg, 55 mg, 60 mg, 65 mg, 70 mg, 75 mg, 80 mg, 85 mg, 90 mg, 95 mg or 100 mg of 1 may be administered per single dose. In the event that acid addition salts or other salts of 1 are used, the corresponding amount of salt used can easily be calculated by the skilled man from the values given hereinbefore, depending on the choice of acid.

If telmisartan 1 is administered in conjunction with an anticholinergic 2, the amount of anticholinergic used will fluctuate considerably depending on the choice of active substance.

Without restricting the invention thereto, in the case of tiotropium 2.1′ amounts of anticholinergic 2 may be administered such that each single dose contains 0.1-80 μg, preferably 0.5-60 μg, particularly preferably about 1-50 μg of 2.1′. For example and without restricting the present invention thereto, 2.5 μg, 5 μg, 10 μg, 18 μg, 20 μg, 36 μg or 40 μg 2.1′ may be administered per single dose. The corresponding amount of salt 2.1 or of any hydrate or solvate used in each case can easily be calculated by the skilled man, depending on the choice of anion. If for example tiotropium bromide is used as the preferred tiotropium salt 2.1 according to the invention, the amounts of the active substance 2.1′ administered per single dose as specified by way of example hereinbefore correspond to the following amounts of 2.1 administered per single dose: 3 μg, 6 μg, 12 μg, 21.7 μg, 24.1 μg, 43.3 μg and 48.1 μg 2.1. In the case of tiotropium 2.1′ the dosages specified above are preferably administered once or twice a day, while administration once a day is particularly preferred according to the invention.

Without restricting the invention thereto, in the case of the cation 2.2′ amounts of anticholinergic 2 may be administered such that each single dose contains 1-500 μg, preferably 5-300 μg, particularly preferably 15-200 μg 2.2′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2.2′ may be administered per single dose. The corresponding amount of salt 2.2 used in each case or of any hydrate or solvate used can easily be calculated by the skilled man, depending on the choice of anion. In the case of oxitropium 2.2′ the dosages specified above are preferably administered one to four times a day, while administration two to three times a day is particularly preferred according to the invention.

Without restricting the invention thereto, in the case of the cation 2.3′ amounts of anticholinergic 2 may be administered such that each single dose contains 1-500 μg, preferably 5-300 μg, particularly preferably 15-200 μg 2.3′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2.3′ may be administered per single dose. The corresponding amount of salt 2.3 used in each case or of any hydrate or solvate used can easily be calculated by the skilled man, depending on the choice of anion. In the case of flutropium 2.3′ the dosages specified above are preferably administered one to four times a day, while administration two to three times a day is particularly preferred according to the invention.

Without restricting the invention thereto, in the case of the cation 2.4′ amounts of anticholinergic 2 may be administered such that each single dose contains 1-500 μg, preferably 5-300 μg, particularly preferably 20-200 μg 2.4′. For example and without restricting the present invention thereto, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2.4′ may be administered per single dose. The corresponding amount of salt 2.4 used in each case or of any hydrate or solvate used can easily be calculated by the skilled man, depending on the choice of anion. In the case of ipratropium 2.4′ the dosages specified above are preferably administered one to four times a day, while administration two to three times a day, more preferably three times a day, is particularly preferred according to the invention.

Without restricting the invention thereto, in the case of the cation 2.5′ amounts of anticholinergic 2 may be administered such that each single dose contains 1-500 μg, preferably 5-300 μg, particularly preferably 15-200 μg. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2.5′ may be administered per single dose. The corresponding amount of salt 2.5 used in each case or of any hydrate or solvate used can easily be calculated by the skilled man, depending on the choice of anion. In the case of glycopyrronium 2.5′ the dosages specified above are preferably administered one to four times a day, while administration two to three times a day is particularly preferred according to the invention.

Without restricting the invention thereto, in the case of the cation 2.6′ amounts of anticholinergic 2 may be administered such that each single dose contains 1000-6500 μg, preferably 2000-6000 μg, particularly preferably 3000-5500 μg, particularly preferably 4000-5000 μg 2.6′. For example and without restricting the present invention thereto, 3500 μg, 3750 μg, 4000 μg, 4250 μg, 4500 μg, 4750 μg, or 5000 μg of 2.6′ may be administered per single dose. The corresponding amount of salt 2.6 used in each case or of any hydrate or solvate used can easily be calculated by the skilled man, depending on the choice of anion. In the case of trospium 2.6′ the dosages specified above are preferably administered one to four times a day, while administration two to three times a day is particularly preferred according to the invention.

Without restricting the invention thereto, in the case of the cation 2.7′ amounts of anticholinergic 2 may be administered such that each single dose contains 50-1000 μg, preferably 100-800 μg, particularly preferably 200-700 μg, particularly preferably 300-600 μg 2.7′. For example and without restricting the present invention thereto, 300 μg, 350 μg, 400 μg, 450 μg, 500 μg, 550 μg, or 600 μg of 2.7′ may be administered per single dose. The corresponding amount of salt 2.7 used in each case or of any hydrate or solvate used can easily be calculated by the skilled man, depending on the choice of anion. In the case of the cation 2.7′ the dosages specified above are preferably administered one to three times a day, while administration once or twice a day, more preferably once a day, is particularly preferred according to the invention.

Without restricting the invention thereto, in the case of the cations 2.9′ and 2.10′, amounts of anticholinergic 2 may be administered such that each single dose contains 1-500 μg, preferably 5-300 μg, particularly preferably 15-200 μg 2.9′ or 2.10′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2.9′ or 2.10′ may be administered per single dose. The corresponding amount of salt 2.9′ or 2.10′ or of any hydrate or solvate used in each case can easily be calculated by the skilled man, depending on the choice of anion. In the case of the cations 2.9′ or 2.10′ the dosages specified above are preferably administered one to three times a day, while administration once or twice a day, more preferably once a day, is particularly preferred according to the invention.

Without restricting the invention thereto, in the case of the cations 2.11′ to 2.13′ amounts of anticholinergic 2 may be administered such that each single dose contains 1-500 μg, preferably 5-300 μg, particularly preferably 10-200 μg 2.11′, 2.12′ or 2.13′. For example and without restricting the present invention thereto, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2.11′, 2.12′ or 2.13′ may be administered per single dose. The corresponding amount of salt 2.11, 2.12 or 2.13 or of any hydrate or solvate used in each case can easily be calculated by the skilled man, depending on the choice of anion.

In the case of the cations 2.11, 2.12 or 2.13 the dosages specified above are preferably administered one to three times a day, while administration once or twice a day, more preferably once a day, is particularly preferred according to the invention.

The aforementioned examples of possible doses applicable for the combinations according to the invention are to be understood as referring to doses per single application. However, these examples are not be understood as excluding the possibility of administering the combinations according to the invention multiple times. Depending on the medical need patients may receive also multiple inhalative applications. As an example patients may receive the combinations according to the invention for instance two or three times (e.g. two or three puffs with a powder inhaler, an MDI etc) in the morning of each treatment day. As the aforementioned dose examples are only to be understood as dose examples per single application (i.e. per puff) multiple application of the combinations according to the invention leads to multiple doses of the aforementioned examples. The application of the compositions according to the invention can be for instance once a day, or depending on the duration of action of the anticholinergic agent twice a day, or once every 2 or 3 days.

Moreover it is emphazised that the aforementioned dose examples are to be understood as examples of metered doses only. In other terms, the aforementioned dose examples are not to be understood as the effective doses of the combinations according to the invention that do in fact reach the lung. It is clear for the person of ordinary skill in the art that the delivered dose to the lung is generally lower than the metered dose of the administered active ingredients.

Telmisartan 1 may be administered in each case by inhalation or by oral, parenteral or some other route, in known manner, in substantially conventional formulations such as for example plain or coated tablets, pills, granules, aerosols, syrups, emulsions, suspensions, powders and solutions, using inert, non-toxic, pharmaceutically suitable carriers or solvents.

In combinations of 1 and 2 the active substance components 1 and 2 may be administered—together or separately—in each case by inhalation or by oral, parenteral or some other route, in known manner, in substantially conventional formulations such as for example plain or coated tablets, pills, granules, aerosols, syrups, emulsions, suspensions, powders and solutions, using inert, non-toxic, pharmaceutically suitable carriers or solvents.

Suitable preparations for administering telmisartan 1 (optionally combined with 2) include tablets, capsules, suppositories, solutions, powders, etc. The proportion of pharmaceutically active compound or compounds should be in the range from 0.05 to 90% by weight, preferably 0.1 to 50% by weight of the total composition. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations of active substances according to the invention may additionally contain a sweetener such as saccharine, cyclamate, glycerol or sugar and a flavour enhancer, e.g. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

In a preferred embodiment the component 2 is administered by inhalation while 1 may also be administered in a manner outlined hereinbefore. In another embodiment 1 and 2 are both administered by inhalation, possibly but not necessarily in a single preparation containing the active substances 1 and 2 or by means of separate preparations each containing only one of the active substances 1 and 2 suitable for administration by inhalation.

Inhalable preparations comprising 2 alone or optionally combinations thereof with 1 include inhalable powders, propellant-containing metered dose aerosols or propellant-free inhalable solutions. Inhalable powders according to the invention containing the active substance(s) 2 and optionally 1 may consist of the active substance on their own or of a mixture of the active substances with physiologically acceptable excipients. Within the scope of the present invention, the term propellant-free inhalable solutions also includes concentrates or sterile inhalable solutions ready for use. The preparations according to the invention may contain the active substance(s) 2 and optionally 1 either together in one formulation or in two separate formulations.

Formulations for inhalation which may be used within the scope of the present invention are described in more detail in the next part of the specification.

The inhalable powders according to the invention may contain 2 and optionally 1 either on their own or in admixture with suitable physiologically acceptable excipients. If the active substances are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextrans), polyalcohols (e.g. sorbitol, mannitol, xylitol), salts (e.g. sodium chloride, calcium carbonate) or mixtures of these excipients with one another. Preferably, mono- or disaccharides are used, while the use of lactose, trehalose or glucose is preferred, particularly, but not exclusively, in the form of their hydrates.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronised active substance preferably with an average particle size of 0.5 to 10 μm, more preferably from 1 to 6 μm, is added to the excipient mixture. Processes for producing the inhalable powders according to the invention by grinding and micronising and finally mixing the ingredients together are known from the prior art. The inhalable powders according to the invention may be administered using inhalers known from the prior art. Inhalable powders according to the invention which contain a physiologically acceptable excipient in addition to 2 and optionally 1 may be administered, for example, by means of inhalers which deliver a single dose from a supply using a measuring chamber as described in U.S. Pat. No. 4,570,630A, or by other means as described in DE 36 25 685 A. The inhalable powders according to the invention which contain 2 and optionally 1 optionally in conjunction with a physiologically acceptable excipient may be administered, for example, using the inhaler known by the name Turbuhaler® or using inhalers as disclosed for example in EP 237507A. Preferably, the inhalable powders according to the invention which contain physiologically acceptable excipient in addition to 2 and optionally 1 are packed into capsules (to produce so-called inhalettes) which are used in inhalers as described, for example, in WO 94/28958.

A particularly preferred inhaler for using the pharmaceutical combination according to the invention in capsules is known from WO 03/084502 (cf. in particular FIG. 1). This inhaler (Handihaler®) for inhaling powdered pharmaceutical compositions from capsules is characterised by a housing 1 containing two windows 2, a deck 3 in which there are air inlet ports and which is provided with a screen 5 secured by a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, and air through-holes 13 for adjusting the flow resistance.

If the inhalable powders according to the invention are to be packaged in capsules, in accordance with the preferred method of administration described above, the capsules should preferably contain from 1 to 30 mg each. According to the invention they contain either together or separately the dosages per single dose specified for 1 and 2 hereinbefore.

Inhalation aerosols containing propellant gas according to the invention may contain substances 2 and optionally 1 dissolved in the propellant gas or in dispersed form. 2 and optionally 1 may be present in separate formulations or in a single preparation, in which 2 and optionally 1 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as preferably chlorinated and fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane), TG227 (1,1,1,2,3,3,3-heptafluoropropane) and mixtures thereof, the propellant gases TG134a, TG227 and mixtures thereof being preferred.

The propellant-driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of 2 and optionally telmisartan 1. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of 2 and optionally telmisartan 1.

If the active substances 2 and optionally 1 are present in dispersed form, the particles of active substance preferably have an average particle size of up to 10 μm, preferably from 0.1 to 6 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention.

The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

Propellant-free inhalable solutions according to the invention contain for example aqueous or alcoholic, preferably ethanolic solvents, possibly ethanolic solvents in admixture with aqueous solvents. In the case of aqueous/ethanolic solvent mixtures the relative proportion of ethanol to water is not restricted, but the maximum limit is up to 70 percent by volume, more particularly up to 60 percent by volume of ethanol. The remainder of the volume is made up of water. The solutions or suspensions containing 2 and optionally 1 separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid, etc. Preferred inorganic acids are hydrochloric acid and sulphuric acid. It is also possible to use the acids which have already formed an acid addition salt with one of the active substances. Of the organic acids, ascorbic acid, fumaric acid and citric acid are preferred. If desired, mixtures of the above acids may also be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabiliser or complexing agent is unnecessary in the present formulation. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the active substances 2 and optionally 1 only benzalkonium chloride and sodium edetate. In another preferred embodiment, no sodium edetate is present.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 and 30 μL of active substance solution can be nebulised in preferably one spray action to form an aerosol with an average particle size of less than 20 μm, preferably less than 10 μm, such that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are known by the name Respimat®.

The Examples which follow serve to illustrate the present invention in more detail without restricting the scope of the invention to the following embodiments by way of example.

### EXAMPLES OF FORMULATIONS

The combinations according to the invention may be administered simultaneously or successively. If the compositions are administered successively telmisartan 1 is preferably administered orally. Preferred oral compositions containing telmisartan 1 are depicted below:

### Example 1

| Constituent | mg per tablet | mg per tablet |
|---|---|---|
| Telmisartan | 40.000 | 80.000 |
| Sodium hydroxide | 3.360 | 6.720 |
| Povidone | 12.000 | 24.000 |
| Meglumine | 12.000 | 24.000 |
| Sorbitol | 168.640 | 337.280 |
| Magnesium stearate | 4.000 | 8.000 |
| Total | 240.000 | 480.000 |

### Example 2

| Constituent | mg per tablet | % of tablet |
|---|---|---|
| Telmisartan | 40.000 | 23.529 |
| Poloxamer | 8.000 | 4.706 |
| Meglumine | 40.000 | 23.529 |
| Mannitol | 80.500 | 47.353 |
| Magnesium stearate | 1.500 | 0.883 |
| Total | 170.000 | 100.000 |

### Example 3

| Constituent | mg per tablet | % of tablet |
|---|---|---|
| Telmisartan sodium salt | 83.417 | 17.379 |
| Sorbitol | 389.383 | 81.121 |
| Magnesium stearate | 7.200 | 1.500 |
| Total | 480.000 | 100.000 |

The anticholinergic 2 is preferably administered via inhalation. Possible examples of inhalable formulations for 2 are specified below.

### Inhalable Powders:

1)

| Ingredients | μg per capsule |
|---|---|
| tiotropium bromide | 10.8 |
| lactose | 4989.2 |
| Total | 5000 |

2)

| Ingredients | μg per capsule |
|---|---|
| tiotropium bromide | 21.7 |
| lactose | 4978.3 |
| Total | 5000 |

3)

| Ingredients | μg per capsule |
|---|---|
| tiotropium bromide × H₂O | 22.5 |
| lactose | 4977.5 |
| Total | 5000 |

4)

| Ingredients | μg per capsule |
|---|---|
| scopine 2,2-diphenylpropionic | 200 |
| acid ester methobromide | |
| Lactose | 23000 |
| Total | 25000 |

5)

| Ingredients | μg per capsule |
|---|---|
| scopine 2,2-diphenylpropionic | 100 |
| acid ester methobromide | |
| Lactose | 12400 |
| Total | 12500 |

6)

| Ingredients | μg per capsule |
|---|---|
| scopine 2,2-diphenylpropionic | 50 |
| acid ester methobromide | |
| Lactose | 4950 |
| Total | 5000. |

7)

| Ingredients | μg per capsule |
|---|---|
| tropenol 2,2- | 200 |
| diphenylpropionic acid ester | |
| methobromide | |
| Lactose | 24800 |
| Total | 25000 |

8)

| Ingredients | μg per capsule |
|---|---|
| scopine 3,3′,4,4′- | 100 |
| tetrafluorobenzilic acid ester | |
| methobromide | |
| Lactose | 12400 |
| Total | 12500 |

9)

| Ingredients | μg per capsule |
|---|---|
| scopine 4,4′- | 100 |
| tetrafluorobenzilic acid ester | |
| methobromide | |
| Lactose | 12400 |
| Total | 12500 |

10)

| Ingredients | μg per capsule |
|---|---|
| tropenol 4,4′- | 100 |
| tetrafluorobenzilic acid ester | |
| methobromide | |
| Lactose | 12400 |
| Total | 12500 |

11)

| Ingredients | μg per capsule |
|---|---|
| 2.7-en (bromide) | 150 |
| Lactose | 12350 |
| Total | 12500 |

12)

| Ingredients | μg per capsule |
|---|---|
| 2.7-en (bromide) | 200 |
| Lactose | 24800 |
| Total | 25000 |

13)

| Ingredients | μg per capsule |
|---|---|
| scopine 9-methyl-fluorene-9- | 80 |
| carboxylate methobromide | |
| Lactose | 12408 |
| Total | 12500 |

14)

| Ingredients | μg per capsule |
|---|---|
| scopine 9-methyl-fluorene-9- | 30 |
| carboxylate methobromide | |
| Lactose | 12420 |
| Total | 12500 |

15)

| Ingredients | μg per capsule |
|---|---|
| cyclopropyltropine 9-hydroxy- | 80 |
| xanthene-9-carboxylate | |
| methobromide | |
| Lactose | 12370 |
| Total | 12500 |

16)

| Ingredients | μg per capsule |
|---|---|
| scopine 9-methyl-fluorene-9- | 100 |
| carboxylate methobromide | |
| Lactose | 24875 |
| Total | 25000 |

17)

| Ingredients | μg per capsule |
|---|---|
| scopine 9-methyl-fluorene-9- | 24 |
| carboxylate methobromide | |
| Lactose | 4964 |
| Total | 5000 |

18)

| Ingredients | μg per capsule |
|---|---|
| tropenol 9-methyl-fluorene-9- | 24 |
| carboxylate methobromide | |
| Lactose | 4964 |
| Total | 5000 |

19)

| Ingredients | μg per capsule |
|---|---|
| tropenol 9-methyl-fluorene-9- | 80 |
| carboxylate methobromide | |
| Lactose | 12408 |
| Total | 12500 |

20)

| Ingredients | μg per capsule |
|---|---|
| tropenol 9-methyl-fluorene-9- | 30 |
| carboxylate methobromide | |
| Lactose | 12420 |
| Total | 12500 |

21)

| Ingredients | μg per capsule |
|---|---|
| tropenol 9-methyl-fluorene-9- | 100 |
| carboxylate methobromide | |
| Lactose | 24875 |
| Total | 25000 |

### B) Propellant-Containing Inhalable Aerosols:

22)

| Ingredients | % by weight |
|---|---|
| scopine 9-methyl-fluorene-9- | 0.010 |
| carboxylate methobromide | |
| Soya lecithin | 0.2 |
| TG 134a:TG 227 = 2:3 | ad 100 |

23)

| Ingredients | % by weight |
|---|---|
| scopine 9-methyl-fluorene-9- | 0.030 |
| carboxylate methobromide | |
| absolute ethanol | 0.5 |
| Isopropyl myristate | 0.1 |
| TG 227 | ad 100 |

24)

| Ingredients | % by weight |
|---|---|
| scopine 9-methyl-fluorene-9- | 0.010 |
| carboxylate methobromide | |
| Soya lecithin | 0.2 |
| TG 134a:TG 227 = 2:3 | ad 100 |

25)

| Ingredients | % by weight |
|---|---|
| tiotropium bromide | 0.015 |
| soya lecithin | 0.2 |
| TG 134a:TG 227 = 2:3 | ad 100 |

26)

| Ingredients | % by weight |
|---|---|
| tiotropium bromide | 0.029 |
| absolute ethanol | 0.5 |
| isopropyl myristate | 0.1 |
| TG 227 | ad 100 |

27)

| Ingredients | % by weight |
|---|---|
| tiotropium bromide | 0.042 |
| absolute ethanol | 30 |
| purified water | 1.5 |
| anhydrous citric acid | 0.002 |
| TG 134a | ad 100 |

28)

| Ingredients | % by weight |
|---|---|
| scopine 2,2-diphenylpropionic | 0.020 |
| acid ester methobromide | |
| Soya lecithin | 0.2 |
| TG 11:TG12 = 2:3 | ad 100 |

29)

| Ingredients | % by weight |
|---|---|
| scopine 2,2-diphenylpropionic | 0.039 |
| acid ester methobromide | |
| absolute ethanol | 0.5 |
| Isopropyl myristate | 0.1 |
| TG 227 | ad 100 |

30)

| Ingredients | % by weight |
|---|---|
| tropenol 2,2- | 0.020 |
| diphenylpropionic acid ester | |
| methobromide | |
| Soya lecithin | 0.2 |
| TG 11:TG12 = 2:3 | ad 100 |

31)

| Ingredients | % by weight |
|---|---|
| tropenol 2,2- | 0.039 |
| diphenylpropionic acid ester | |
| methobromide | |
| absolute ethanol | 0.5 |
| Isopropyl myristate | 0.1 |
| TG 227 | ad 100 |

32)

| Ingredients | % by weight |
|---|---|
| tropenol 9-methyl-fluorene-9- | 0.050 |
| carboxylate methobromide | |
| Soya lecithin | 0.2 |
| TG 134a:TG 227 = 2:3 | ad 100 |

33)

| Ingredients | % by weight |
|---|---|
| tropenol 9-methyl-fluorene-9- | 0.080 |
| carboxylate methobromide | |
| absolute ethanol | 0.5 |
| Isopropyl myristate | 0.1 |
| TG 227 | ad 100 |

34)

| Ingredients | % by weight |
|---|---|
| tropenol 9-methyl-fluorene-9- | 0.050 |
| carboxylate methobromide | |
| Soya lecithin | 0.2 |
| TG 134a:TG 227 = 2:3 | ad 100 |

1. A pharmaceutical composition comprising telmisartan 1, optionally in form of the salts, solvates or hydrates thereof, and an anticholinergics 2, optionally in the form of the solvates or hydrates thereof.

2. The pharmaceutical composition according to claim 1, wherein the anticholinergic (2) is selected from the group comprising tiotropium salts (2.1), oxitropium salts (2.2), flutropium salts (2.3), ipratropium salts (2.4), glycopyrronium salts (2.5), trospium salts (2.6), an anticholinergic of formula 2.7 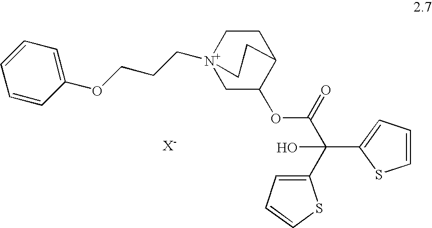
wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
optionally in the form of the racemates, enantiomers or hydrates thereof,
and an anticholinergic of formula 2.8 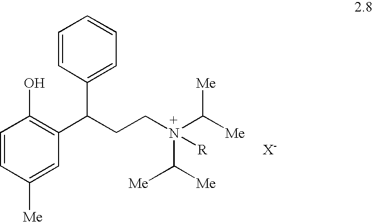
wherein R denotes either methyl (2.8.1) or ethyl (2.8.2) and wherein X⁻ may have the meanings mentioned hereinbefore, optionally in the form of the racemates, enantiomers or hydrates thereof.

3. Pharmaceutical compositions according to claim 1, wherein the anticholinergic (2) is selected from anticholinergics of formula 2.9 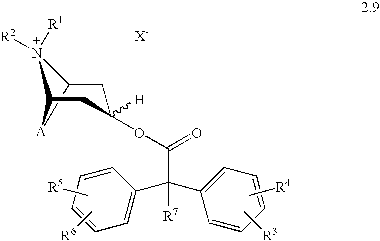
wherein
A denotes a double-bonded group selected from the groups 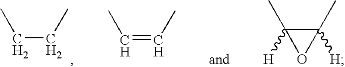
X⁻ denotes an anion with a single negative charge;
R¹ and R² which may be identical or different denote a group selected from methyl, ethyl, n-propyl and iso-propyl, which may optionally be substituted by hydroxy or fluorine, preferably unsubstituted methyl;
R³, R⁴, R⁵ and R⁶, which may be identical or different, denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—CH₂—F, —O—CH₂—F, —O—CH₂—CH₂—F, —CH₂—OH, —CH₂—CH₂—OH, CF₃, —CH₂—OMe, —CH₂—CH₂—OMe, —CH₂—OEt, —CH₂—CH₂—OEt, —O—COMe, —O—COEt, —O—COCF₃, —O—COCF₃, fluorine, chlorine or bromine,
optionally in the form of the racemates, enantiomers or hydrates thereof.

4. The pharmaceutical composition according to claim 1, wherein the anticholinergic g is selected from the compounds of formula 2.10 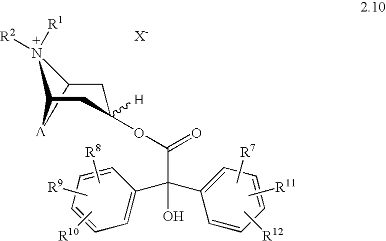
wherein
A, X⁻, R¹ and R² may have the meanings given in claim 4 and wherein
R⁷, R⁸, R⁹, R¹⁰, R¹¹ and R¹², which may be identical or different, denote hydrogen, methyl, ethyl, methyloxy, ethyloxy, hydroxy, fluorine, chlorine, bromine, CN, CF₃ or NO₂, while at least one of the groups R⁷, R⁸, R⁹, R¹⁰, R¹¹ and R¹² may not be hydrogen,
optionally in the form of the racemates, enantiomers or hydrates thereof.

5. The pharmaceutical compositions according to claim 1, wherein the anticholinergic (2) is selected from the compounds of formula 2.11 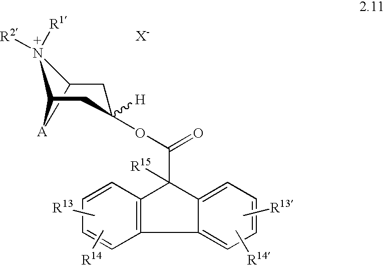
wherein
A and X⁻ may have the meanings given in claim 4 and wherein
R¹⁵ denotes hydrogen, hydroxy, methyl, ethyl, —CF₃, CHF₂ or fluorine;
R^{1′} and R^{2′} which may be identical or different, denote C₁-C₅-alkyl, which may optionally be substituted by C₃-C₆-cycloalkyl, hydroxy or halogen,
or
R^{1′} and R^{2′} together denote a —C₃-C₅-alkylene bridge;
R¹³, R¹⁴, R^{13′} and R^{14′} which may be identical or different, denote hydrogen, —C₁-C₄-alkyl, —C₁-C₄-alkyloxy, hydroxy, —CF₃, —CHF₂, CN, NO₂ or halogen,
optionally in the form of the racemates, enantiomers or hydrates thereof.

6. The pharmaceutical compositions according to claim 1, wherein the anticholinergic (2) is selected from anticholinergics of formula 2.12 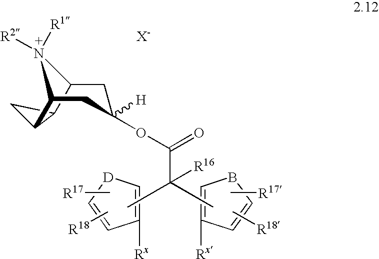
wherein X⁻ may have the meanings given in claim 4 and wherein
D and B which may be identical or different, preferably identical, denote O, S, NH, CH₂, CH═CH or N(C₁-C₄-alkyl);
R¹⁶ denotes hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃ or halogen;
R^{1″} and R^{2″} which may be identical or different, denote —C₁-C₅-alkyl, which may optionally be substituted by —C₃-C₆-cycloalkyl, hydroxy or halogen,
or
R^{1″} and R^{2″} together denote a —C₃-C₅-alkylene bridge;
R¹⁷, R¹⁸, R^{17′} and R^{18′}, which may be identical or different, denote hydrogen, —C₁-C₄-alkyl, —C₁-C₄-alkyloxy, hydroxy, —CF₃, —CHF₂, CN, NO₂ or halogen;
R^{x} and R^{x′} which may be identical or different, denote hydrogen, —C₁-C₄-alkyl, —C₁-C₄-alkyloxy, hydroxy, —CF₃, —CHF₂, CN, NO₂ or halogen,
or
R^{x} and R^{x′} together denote a single bond or one of the double-bonded groups O, S, NH, CH₂, CH₂—CH₂, N(C₁-C₄-alkyl), CH(C₁-C₄-alkyl) and —C(C₁-C₄-alkyl)₂,
optionally in the form of the racemates, enantiomers or hydrates thereof.

7. The pharmaceutical compositions according to claim 1, wherein the anticholinergic (2) is selected from anticholinergics of formula 2.13 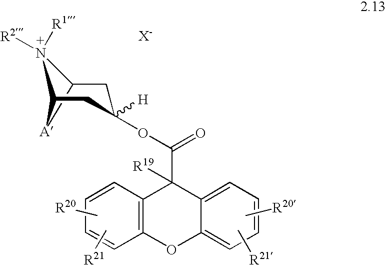
wherein X⁻ may have the meanings given in claim 4 and wherein
A′ denotes a double-bonded group selected from 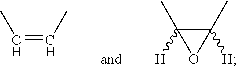
R¹⁹ denotes hydroxy, methyl, hydroxymethyl, ethyl, —CF₃, CHF₂ or fluorine;
R^{1″} and R^{2″} which may be identical or different, denote C₁-C₅-alkyl, which may optionally be substituted by C₃-C₆-cycloalkyl, hydroxy or halogen,
or
R^{1″} and R^{2″} together denote a —C₃-C₅-alkylene bridge;
R²⁰, R²¹, R^{20′} and R^{21′} which may be identical or different, denote hydrogen, —C₁-C₄-alkyl, —C₁-C₄-alkyloxy, hydroxy, —CF₃, —CHF₂, CN, NO₂ or halogen,
optionally in the form of the racemates, enantiomers or hydrates thereof.
